(19) United States
(12) Patent Application Publication
(10) Pub. No.: US 2003/0149595 A1
(43) Pub. Date: Aug. 7, 2003

(54) Clinical bioinformatics database driven pharmaceutical system

(76) Inventor: John E. Murphy, Atlanta, GA (US)

Correspondence Address: DORSEY & WHITNEY, LLP, INTELLECTUAL PROPERTY DEPARTMENT, 370 SEVENTEENTH STREET, SUITE 4700, DENVER, CO, 80202-5647, US

(21) Appl. No.: 10/166,318

(22) Filed: Feb. 1, 2002

(51) Int. Cl. G06F 17/60; G06F 19/00; G01N 33/48; G01N 33/50

(52) U.S. Cl. 705/2; 702/19

(57) ABSTRACT

Computer-based technologies and methods of human clinical data capture and analysis for identifying and recruiting patients for pharmaceutical and diagnostic product testing. These methods include acquiring product data and clinical data and comparing product data to clinical data in real time in order to identify suitable patients for product testing. Methods also provide for the generation of an alert message identifying suitable patients, preferably through the use of artificial intelligence or neural network techniques. Methods also preferably include the use of wireless devices to collect the patient data with a graphical user interface suitable of displaying the alert message and receiving additional questions for use in querying the patients for collection of data, the encryption of clinical data during transmission and storage, and conversion of clinical data to a format consistent with data mining techniques.

### OVERVIEW

Predict Incorporated is a clinical bioinformatics company that provides Very Large Scale Clinical Databases (VLSCD) and Automated Artificially Intelligence Data to Knowledge Conversion for the pharmaceutical industry to expedite the trial and introduction of new drugs to market. The company has developed a powerful set of software that allows it to collect and analyze large volumes of real-time clinical information to provide very specific prediction of how a patient will respond to a given drug compound. These techniques speed up the way that drugs can be designed and tested and ultimately will change the way that doctors diagnose and treat disease. The value of Predict's technology can be measured in the hundreds of millions of dollars per year that will be saved in the drug discovery process and the billions of dollars per year in new drugs entering the pipeline.

The terms “BioSolomon” and “Springfree” are trademarks of Predict Inc.

### The Reason for Clinical Bioinformatics

Physicians and pharmaceutical researchers have long known that genetic alterations can lead to disease. Mutations in one gene cause cystic fibrosis; in another gene, sickle cell anemia. But through the work of academic research centers around the world and corporations such as Celera and Human Genome Sciences, it is now clear that genetic differences between individuals can also affect how well a person absorbs, breaks down (metabolizes) and responds to various drugs. The cholesterol-lowering drug pravastatin, for example, does nothing for people with high cholesterol who have a common variant of an enzyme called cholesteryl transfer protein.

Genetic variations can also render drugs toxic to certain individuals. Isoniazid, a tuberculosis drug, causes tingling, pain and weakness in the limbs of those who are termed slow acetylators. These individuals possess a less active form of the enzyme N-acetyltransferase, which normally helps clear the drug from the body. Thus, the drug can outlive its usefulness and may stick around long enough to get in the way of other, normal biochemical processes. If slow acetylators receive procainamide, a drug commonly given after a heart attack, they stand a good chance of developing an autoimmune disease resembling lupus.

In recent years much attention has been given to “cancer genes”—the so called oncogenes—that has led to a widespread misconception that we all carry around cancercausing genes in our cells; but this is not so. The genes in question are entirely normal and necessary for life. They are, however, potential cancer genes, or proto-oncogenes, because after undergoing certain abnormal changes in their genetic sequence, the modified genes turn a cell cancerous. The change can be a point mutation within a gene—as simple as substituting one DNA base for another—or it can be a rearrangement within the gene, or it can be the accidental pairing of a gene with a regulatory sequence that drives the normal gene faster than normal. Whatever the change, it is now clear from research studies that one alteration or mutation is not enough. Several genes—as few as two in one form of cancer to perhaps ten or twenty in other types—must be changed to transform a well-behaved cell into a rampaging killer. If the right mutations occur, a cell will surely become cancerous, but those changes come at the end of a long and improbable chain of causation.

Before cancer can start, a whole series of rare events must occur. The cancer process starts in many people through contact with cancer causing substances, or carcinogens, such as benzopyrene, found in tobacco smoke. Contrary to popular impression, however, chemical carcinogens are not always harmful in their original form. These substances arrive in the body innocuous and are turned into potential killers by the body itself. Specialized cells whose job is supposed to be to detoxify poisons that get into the body in the liver, skin, lymphatic system and other organs chemically alter the unwanted molecules into a form that is more easily excreted. Researchers at the National Cancer Institute have found however, that people differ genetically in their complement of detoxifying enzymes. Errant enzymes sometimes perform the wrong modification to carcinogenic molecules. Instead of rendering them harmless, the enzymes alter the molecule so that it becomes more potent—better at slipping into a cell's nucleus; or more avid in its ability to bind to DNA in a way that affects a gene's activity. This modification of the carcinogen, called activation, is the first step toward a cancer-causing mutation.

Cancers are most common among cells that have a high rate of cell cycling. More than 90 percent of all cancers in adults arise in just one type of tissue, the epithelial cells that make up skin and the lining of the gastrointestinal tract, the uterus, the lungs and airways and the glands. Cancer is extremely rare among cell types that never divide. Substances that speed cell replacement are likely to be carcinogenic because they increase rates of cell proliferation and cell death. Substances that accelerate cell division are known as promoters and work in concert with proto-oncogenes to cause cancer. Phenobarbital is a strong promoter of liver cells. Cigarette tar contains promoters that speed the proliferation of lung cells. Saccharine and cyclamate are each weak carcinogens, but strong promoters. Even some mechanical processes, such as skin abrasion can act as promoters.

In 1989, the Nobel Prize for medicine was awarded to two researchers who began to shape the modem view of cancer as a genetic disease—a result of derangement in DNA. DNA within a cell provides the instruction set that allows the cell to perform its normal function through the production of proteins. Think, for example, of the genes whose protein products help regulate the cell cycle. Such a protein might tell the cell to divide under specific circumstances. Imagine, now, that the gene is damaged so that its protein no longer waits for some outside signal but constantly tells the cell to divide. Just such an oncogene, called ras, has been found in a considerable number of human tumor cells. The normal form of the protein resides just inside the cell membrane and has the characteristics of the molecules whose job is to relay signals brought by proteins arriving at receptors on the cell surface. It appears that the mutant ras simply relays a signal even when nothing has arrived at the receptor, so the cell divides continuously.

Several other proto-oncogenes, to cite other roles, contain codes for enzymes that attach phosphates to specific sites or proteins. This process, called phosphorylation is one of the most powerful regulatory mechanisms within cells. When proteins are phosphorylated, they change their shape and their biochemical powers. When the same proteins are dephosphorylated, the shapes and powers change back to the original. Many of the metabolic steps essential to life are governed through this process. Many oncogenes, it turns out, are genes for enzymes that phosphorylate various specific proteins. Such molecules are called protein kinases. Since a single type of protein kinase may phosphorylate several other types of molecules within the cell, a single mutation in one has wide-ranging effects throughout the cell.

Recently, molecular biologists have found another type of cancer gene, one whose history can be much like that of an oncogene but whose normal role is to keep cell division under proper control. If oncogenes are the accelerator pedals of cancer, these genes are protein products that keep the brakes on cell proliferation. If one of these genes is damaged, the brakes are released and the cell automatically leaps into high gear. Such genes are called tumor suppressor genes. The best-known tumor suppressor gene carries the label p53 (protein with a molecular weight of 53 daltons) and is known to play a role in about fifty percent of all human cancers. p53 stimulates DNA inspection and repair enzymes and prevents the cell from replicating its chromosomes until all necessary quality control and repair processes have been completed. Its core responsibility is to keep a cell with damaged DNA not only from proliferating, but also from continuing to exist at all. p53 acts as a natural born killer for cells that have defective DNA sequences.

When p53 is altered the cell looses its DNA quality control mechanism in the cell division cycle. Without its ability to trigger cell death, cell division is now endowed with cancerous abilities. In 1991, a team of researchers discovered a mechanism by which a carcinogen actually deranged the cell's p53 process. A toxin produced by a fungus that grows in corn, peanuts, and certain other foods known as aflatoxin causes p53 mutation. In half of all liver cancer patients the p53 genes are mutated at the third base in Codon 249. This means that when the cell follows the gene instruction to produce p53 it inserts the wrong amino acid in the 249^{th} position (substituting serine for argenine). Epidemiologists studying liver cancer had noticed that the incidence of liver cancer was uncharacteristically high in South Africa and China, two areas where aflatoxin is common (more about this later).

Over the past twenty years, an understanding of cell and molecular biology has dramatically improved our understanding of the physiology of the cell and how compounds interact with cell membranes, receptors, channels, transport molecules (motor molecules kinesin, dynein, etc.), and basic cell metabolic processes. More recently, the human genome has been mapped and is being deciphered to understand the function of each codon. Celera, the winner of the race to decode the human genome has announced that its next goal is to build the complete library of human protein structures that are created according to DNA blueprint. Proteins are the building blocks of all life processes.

Extraordinary advances in human cell and molecular biology over the past decade have created a wealth of new targets and pathways for drug development that promise cure for cancer, diabetes, heart disease and other major diseases. Unfortunately, this wealth of compound and target and marker knowledge lacks specificity and without a better way to predict which compounds will work best for specific patients and specific disease states the drug industry will continue to invest hundreds of millions of dollars a year on developmental drugs that fail to reach the marketplace because of inconclusivity of effect or side effect. The pharmaceutical industry needs a better method for (1) defining patients for clinical trial; (2) categorizing disease states; (3) cataloging disease promoters; (4) managing clinical trials; (5) tracking iatrogenic and drug side effects; and, (6) marrying clinical data with genomic and proteomic knowledge for the faster production and market approval of new pharmaceuticals. The added market value of such a method can be measured in the billions of dollars per year.

### Clinical Bioinformatics and Pharmacogenomics

Predict Incorporated has developed a proprietary, sophisticated, artificially intelligent computerized data system that provides the drug industry with a powerful scientific way to perform pharmacogenomic, pharmacoproteiomic, DNA promoter forensics, and toxopharmacology. Predict's software collects and analyzes clinical information that is captured at the point-of-care. Its BioSolomon Clinical Data to Knowledge System is structured to house patient histories, exposures, symptoms, vital values, laboratory values including genome-wide analysis of genetic variation, biometry, imaging studies, physical diagnosis, prescriptions and therapies. Patient information can be captured and stored longitudinally in a deidentified, anonymous way to use in a systematic genome-wide analysis to determine which drugs will work best with the fewest side effects in specific categories of patient.

Beyond the promise of improving diagnosis and treatment of disease the Predict BioSolomon Clinical Knowledge System improves the pharmaceutical industries' ability to get more novel drugs to market. Currently 80 percent of drugs in development fail in early clinical trials because they are not effective or are even toxic. To boost the success rate of drug approval the industry needs a way to test new drugs only in individuals who are likely to show benefits from them during the clinical trial. BioSolomon provides the solution to the problem that it is hard to develop drugs that work. The solution simply put is: Predict provides the pharmaceutical industry with a computerized method for generating and testing the largest number of compounds in the shortest amount of time with the least amount of human effort. The Predict system provides a better way for the industry to select the most promising compounds early in the trial process, that is taking a compound and “Fast Forwarding it into Man” in a way that insures the highest probability of success by tightly defining the characteristics of the trial cohort. Being able to test a drug's selectivity, toxicity, metabolism and absorption at the start of the screening process against a select group of patients will cut down on efforts wasted on trying ineffective drugs in broadly defined human trial populations and will save hundreds of millions of dollars per year. Concomitant with the ability to kill bad drugs faster, the Predict BioSolomon Clinical Knowledge System enables the drug industry to predict patient trial cohorts that will most likely benefit from early stage trials. This means that more drugs will enter the pipeline faster, generating billions in new drug sales annually.

### The BioSolomon Data Vault

Predict Incorporated operates a state-of-the-art, fault-tolerant, secure clinical repository that at full deployment will collects real-time clinical data from sites in the United States, Canada, South America, Western Europe, Africa and the Far East via the Internet. The repository manages both relational and object data and through a series of interface engines collects information from legacy point-of-care clinical and laboratory software systems, digital imaging systems and biomedical and biometric devices. This means that the database captures clinical and laboratory data directly from systems produced by Cemer, Sunquest, HBOC-McKesson, Eclypsis, Meditech, and the like, and Single Nucleotide Polymorphisims (SNPs) and genetic information from biometric instruments manufactured by Cytogen, Axcell Bioscience, Ciphergen-Proteomics, Biorad, Zyomix, among others. Patient digital images collected by systems sold by G. E., Phillips and Siemens Corporation, among others are stored along with software motion picture and sound clips. Cardiac and other types of physiologic monitoring are also collected and analyzed. Predict also offers its own wireless, web-based clinical automation software system to clinical sites around the world for maintenance of patient medical records and deidentified clinical information. This Clinical Automation System is integrated with a real-time Predict Pharmaceutical Protocol Software System that directly links the drug industry to clinical sites, anywhere in the world where Predict web-access is available.

Data housed in the BioSolomon Data Vault is analyzed using artificial intelligence data mining techniques where computers evaluate multivariate and multidimensional data to identify clinical facts that are not commonly known. Earlier in this discussion a promoter of disease such as aflatoxin and its action on p53 in oncogenesis was described. This association of a promoter of cancer with diet with specific regions of the world is automatically produced by BioSolomon's heteroassociative neural network. When BioSolomon is coupled with Genomic and Proteomic Databases that have been compiled by research centers such as Lawrence Livermore's Human Genome Center, the Lawrence-Berkley's Genome Institute, the Image Consortium, the John's Hopkins Genome Database, the National Center for Genome Resources, European Biobase, and the Danish Center for Human Genome Research the automation of pharmacogenomics will become a reality. Additional pharmacogenomic and pharmacoproteomic functionality will become available as Predict links BioSolomon with commercial genetic and proteomic databases that are being compiled by companies such as Celera and its Paracel Division, Human Genome Sciences, Incyte Genomics and the other major pharmaceutical houses.

A clinical variable plot can be produced by BioSolomon artificial intelligence. It would show, for example, a universe of patients having a set of clinical information collected by Predict Bioinformatic software. The database includes every data value imaginable, from personal and family illness and exposure histories to social habits, hobbies, medication history, diagnoses, biometric and laboratory values and any other clinical fact that is defined within the system. The level of data granularity in BioSolomon meets known nomenclatures and national and international standards and comprises the complete set of information that clinicians, pharmaceutical companies and the insurance industry might be interested in collecting. It is flexible and additive so that new variables that might be discovered can be easily added.

Today, the typical pharmaceutical industry biostatistician-epidemiologist, when performing research on population samples for drug evaluation, plot variables against an x and a y-axis. Values collected scatter in a distribution across the x and y axes and regression is performed to find a “best-fit” line between the scattered points. Two-dimensional analysis is the best that human computing can reasonably deliver.

BioSolomon A.I. neural networking can however analyze millions of clinical values collected from billions of patients for an unlimited number of variables. This means that the computer can perform “best-fit” analysis in three dimensions and instead of a single dimension regression line can produce a multi-dimensional set of associations showing causal relationships between things such as aflatoxin and p53 on the fly. For Pharmacogenomics-proteomics to succeed BioSolomon is essential and it is unique and not easily duplicated. It can produce a complex three axis data plot representing a best fit for a variety of data (e.g., 50 data elements) collected (e.g., history, symptomatology, lab values, vitals, medications, etc) for 50 patients over 50 days, for example. In two-dimensional analysis this would require an array of combinations and permutations of 50×50×50, or 125,000 separate regressions. BioSolomon can provide answers to complex data questions in minutes that currently take highly trained scientists months to complete using products such as SAS. BioSolomon is capable of providing answers to complex data questions that scientists cannot answer analyze today, because the mathematics would take several life times to complete.

Additional information about exemplary implemenations of the system described above are provided in the Appendices, which are incorporated herein and form a part of this specification. The following identifies the Appendices.

Appendix A: Summary of Bioinformatics System.

Appendix B: Data Vault and Mining System Summary.

Appendix C: System Script Scenario.

Appendix D: Summary of a System Example.

Appendix E: Summary of a System Example.

Appendix F: Summary of a System Example.

Appendix G: Summary of a System Example.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A network-based method for identifying a target group for testing a product on patients, comprising:
acquiring product data for a plurality of parameters relating to testing the product;
acquiring clinical data relating to a plurality of patients;
comparing the product data to the clinical data in order to identify a target group of patients for testing the product;
generating a time parameter relating to a time frame for testing of the product involving the target group; and
providing an indication of the target group and the time parameter.

2. The method of claim 1 wherein the generating step includes providing an alert message concerning identification of a patient satisfying the parameters for the testing.

3. The method of claim 1 wherein the acquiring clinical data step includes providing real-time information relating to the clinical data via the network.

4. The method of claim 1 wherein the acquiring product data step includes identifying parameters for an ideal patient for the testing of the product.

5. The method of claim 1 wherein the comparing step includes using artificial intelligence or neural network techniques in order to identify the target group.

6. The method of claim 1 wherein the acquiring clinical data step includes using a wireless device to acquire the clinical data and transmit the acquired clinical data via a network.

7. The method of claim 1 wherein the acquiring clinical data step includes electronically and automatically acquiring the clinical data and transmitting the acquired clinical data via a network.

8. The method of claim 1, further including displaying a user interface in order to receive the product data.

9. The method of claim 2, further including displaying the alert message within a user interface.

10. The method of claim 2, further including obtaining information relating to parameters for determining when to generate the alert message.

11. The method of claim 1, further including encrypting the clinical data for storage and network transmission.

12. The method of claim 1, further including controlling access to the clinical data.

13. The method of claim 1, further including generating a series of questions for use in querying the patients to obtain the clinical data.

14. The method of claim 1 wherein the comparing step includes identifying the target group for testing of a particular pharmaceutical product.

15. The method of claim 1, further including converting the acquired clinical data to a consistent format for data mining techniques.
